(19) United States
(12) Patent Application Publication
(10) Pub. No.: US 2011/0184239 A1
(43) Pub. Date: Jul. 28, 2011

(54) METHODS AND SYSTEMS FOR DISABLING AN ENDOSCOPE AFTER USE

(75) Inventors: George Wright, Dove Canyon, CA (US); Lonnie Hoyle, Mission Viejo, CA (US); Kais Almarzouk, Tustin, CA (US)

(73) Assignee: INTEGRATED ENDOSCOPY, INC., Lake Forest, CA (US)

(21) Appl. No.: 12/974,452

(22) Filed: Dec. 21, 2010

Related U.S. Application Data
(60) Provisional application No. 61/289,338, filed on Dec. 22, 2009.

(51) Int. Cl. A61B 1/00

(52) U.S. Cl. 600/118

(57) ABSTRACT

Various embodiments comprise endoscopes for viewing inside a cavity of a body such as a vessel like a vein or artery. These endoscopes may include a usage detector and a disabling device coupled to the usage detector, wherein the disabling device is configured to disable the endoscope at least partly in response to an electrical, optical, and/or mechanical output from the usage detector.

### CROSS-REFERENCE TO RELATED APPLICATIONS

The present application claims priority from U.S. Patent Application No. 61/289,338, filed Dec. 22, 2009, the content of which is incorporated herein by reference in its entirety.

### STATEMENT REGARDING FEDERALLY SPONSORED R&D

Not Applicable

### PARTIES OF JOINT RESEARCH AGREEMENT

Not Applicable

### REFERENCE TO SEQUENCE LISTING, TABLE, OR COMPUTER PROGRAM LISTING

Not Applicable

### BACKGROUND

1. Field of the Invention

The present invention relates generally to optical systems, and in some embodiments, to endoscopes and other medical devices.

2. Description of the Related Art

Endoscopes generally include a tube with imaging optics to be inserted into a patient. Illumination may be provided by a source that is located external to the patient. Light from the illumination source may travel via a conduit, such as a fiberoptic or fiberoptic bundle, through the tube into the patient. The light may be emitted inside of the patient at the tube's distal end near a treatment or viewing site. Features inside the body are likewise illuminated and can be viewed using the imaging optics, which form images of the patient's insides.

### SUMMARY OF THE INVENTION

Embodiments of the present invention comprise optical devices, such as endoscopes for viewing inside a cavity of a body such as a vessel like a vein or artery or elsewhere.

Certain embodiments include a mechanism for disabling (optionally permanently disabling) the use of the optical device after a predetermined number of uses (e.g., after a single use, 2 uses, or other pre-specified number of uses). This will ensure that the optical device is not used more often than is intended by the manufacturer or than is safe.

An example embodiment of an endoscope comprises: a usage detector; and a disabling device coupled to the usage detector, wherein the disabling device is configured to disable (optionally permanently disable) the endoscope at least partly in response to an electrical, optical, and/or electrical output from the usage detector.

Another example embodiment of an endoscope comprises: a lens; a usage detector; and a disabling device coupled to the usage detector, wherein the disabling device is configured to disable (optionally permanently disable) the endoscope at least partly in response to: (a) a mechanical output from the usage detector, (b) an optical output from the usage detector, (c) electrical output from the usage detector, or (d) any combination of (a), (b), or (c), wherein the usage detector is configured to detect: (i) an initiation of use of the endoscope, (ii) a termination of use of the endoscope, (iii) a cable insertion, (iv) a cable removal, (v) a switch activation, or (vi) any combination of (i), (ii), (iii), (iv), or (v).

A example method for manufacturing an endoscope for viewing portions of a body is also described, the method comprising: disposing on at least one portion of an endoscope body a usage detector device configured to detect a usage event related to the endoscope, the usage event including one or more of: (i) an initiation of use of the endoscope, (ii) a termination of use of the endoscope, (iii) a cable insertion to the endoscope, (iv) a cable removal from the endoscope, (v) a switch activation, or any combination of (i), (ii), (iii), (iv), or (v); and disposing on at least one portion of an endoscope body a disabling device configured to permanently inhibit the use of the endoscope at least partly in response to: (a) a first mechanical output from a usage detector, (b) a first optical output from the usage detector, (c) a first electrical output from the usage detector, or (d) any combination of (a), (b), or (c).

An example method for operating an endoscope comprises: detecting a usage of the endoscope; and disabling further usage of the endoscope at least partly in response to the detected usage.

Another example method for disabling an endoscope comprises: detecting a usage of the endoscope; disabling the endoscope at least partly in response to: (a) a mechanical output from a usage detector, (b) an optical output from the usage detector, (c) electrical output from the usage detector, or (d) any combination of (a), (b), or (c); and disabling further usage of the endoscope at least partly in response to the detected usage.

### BRIEF DESCRIPTION OF THE DRAWINGS

The disclosed aspects will hereinafter be described in conjunction with the appended drawings, provided to illustrate and not to limit the disclosed aspects, wherein like designations denote the elements.

FIG. 1A illustrates an example endoscope system, including a system disabling device;

FIG. 1B illustrates a first example process;

FIG. 1C illustrates a second example process;

FIG. 1D illustrates an example system for producing images of features inside of body parts;

FIG. 2 illustrates another system for producing images of features inside of body parts;

FIG. 3 is an exploded perspective view of a longitudinal member comprising an endoscope structure;

FIG. 4 is a rear perspective view of an exemplary front lens holder that may be used with the longitudinal member of FIG. 3;

FIG. 5 shows a schematic diagram of an optical path through a front surface tilted at an angle with respect to a rear surface;

FIG. 6 shows another view of a front lens holder for used with a longitudinal member, such as the longitudinal member of FIG. 3;

FIG. 7 is a perspective view of an elongated support structure, which may be used as the cradle of FIG. 3; and

FIG. 8 is a partial perspective view of an exemplary slotted elongate support structure, which may also be used as the cradle of FIG. 3.

### DETAILED DESCRIPTION OF PREFERRED EMBODIMENTS

Embodiments of the present invention comprise endo scopes for viewing inside a cavity of a body such as a vessel like a vein or artery or elsewhere. The present disclosure relates generally to medical devices and methods, and in some embodiments, to endoscopes and other devices for viewing and/or imaging objects inside a body (e.g., a vessel like a vein or artery, a gastrointestinal tract, other cavity, or elsewhere). For the purpose of description, a “body” can be that of a human or non-human animal, and can also be that of a living or non-living animal.

Example endoscopes have a light source that is configured, sized and positioned so as to be inserted into the body cavity to provide illumination therein. In various embodiments, this light source optionally comprises one or more solid state emitters such as a light emitting diode (LED), although other light sources may be used. Optionally, this solid state emitter is small and bright. Light emitted from the light source is reflected off objects or walls in the interior of the body cavity. A portion of the reflected light is collected through an aperture in the endoscope. This light is directed along an optical path through the endoscope so as to form an image of the objects or walls. In certain optional embodiments, the optical path includes a series of lenses such as rod lenses disposed in a support structure or cradle. The light is then directed to an optical sensor such as, for example, an optical detector array or an optical camera (e.g., employing a segmented detector such as a charge-coupled-device (CCD) or a complementary-metal-oxide-semiconductor (CMOS) detector). Thus, an image of the object inside the body cavity can be viewed.

Efforts have been made to reduce the cost of endoscopes so as to make medical procedures more affordable. However, certain techniques used to reduce the cost of an endoscope make the endoscope unsuitable for more than a certain number of uses. For example, some endoscopes are intended to be used only once, and are intended to be disposed of after the single use. Nonetheless, it is anticipated that in certain situations, a doctor or medical facility may attempt to use the endoscope more times than is safe. This may occur accidently or to further save money by improperly reusing the endoscope. Further, certain endoscopes are intended to be used only once and then disposed of in order to eliminate the cost and time associated with the sterilization and to reduce the risk of infection associated with the reuse of the endoscope.

Certain conventional approaches to disabling endoscopes rely on the heat, pressure, or moisture from a sterilization/cleaning process to disable the endoscope. However, such approaches require that such sterilization processes be used. Disadvantageously, such approaches may discourage people that want to improperly reuse the endoscope from using appropriate sterilization processes, so as to allow them to reuse the endoscope. This results in even a more dangerous use of an endoscope that may have been intended and configured for a single use.

Certain embodiments address the foregoing challenge of improper reuse of an endoscope by including a mechanism for disabling (e.g., permanently disabling) the use of the endoscope after a predetermined number of uses (e.g., after a single use, 2 uses, or other pre-specified number of uses). This will ensure that the endoscope is not used more often than is intended by the manufacturer or than is safe for patients.

An example embodiment of the disabling mechanism includes a device for detecting the beginning and/or end of a use of the endoscope. For example, certain embodiments detect (electrically, optically, and/or mechanically) when power is applied to the light source and/or when power is removed from the light source. Upon detecting (e.g., electrically, optically, and/or mechanically) that the endoscope has been used the pre-specified number of times (e.g., one time), further use of the endoscope as an endoscope in prevented and/or inhibited (e.g., by degrading the optical performance to an undesirable level) mechanically, electrically and/or optically.

For example, the disabling mechanism can prevent the light source from illuminating or from illuminating with adequate brightness (e.g., by preventing power from being applied to the light source or reducing the amount of power supplied to the light source) and/or inhibit light from being emitted from the endoscope through an aperture and/or external light (e.g., reflected from internal portions of a patient's body) from being received by the endoscope via the aperture and/or received by the endoscope's optical sensor (e.g., by obscuring some or all of the endoscope aperture, one or more lenses or prisms, and/or the optical sensor). Thus, the disabling mechanism may permanently disable further use of the endoscope, so that the endoscope is not reusable without significant modification of the endoscope (e.g., without opening the endoscope and replacing electrical, optical, and/or mechanical components).

By way of illustration, the act of connecting the endoscope to a power and/or optical cable can be used to prevent reuse of the endoscope. For example, an endoscope can include a pin or other device that prevents a light blocking element (e.g., a physical/mechanical device, such as an opaque or translucent plastic disk, a closeable iris, a leaf shutter, a diaphragm shutter, etc.) from obscuring a light aperture, a face of an internal lens in the light path, or an image sensor, by holding the light blocking element in a non-obscuring position. In addition, the endoscope can include a spring-loaded (e.g., a metal helical spring, a non-coiled spring, a tension/extension spring, a compression spring, a torsional spring, a gas spring, a rubber mount, etc.) connector. The spring urges the connector into a first position. The connector can be used to receive an electrical and/or optical cable. For example, an electrical cable may be used to power the endoscope via a battery or other electrical source. An optical cable can include fiber optic such as a coherent fiber optic bundle.

When a user connects a cable to the connector, the pressure causes the connector to move forward (e.g., slightly forward) against the force exerted by the spring to a second position. As the connector moves forward, it pushes a rod, bar, cam, or other device forward, which in turn pushes the pin into a position such that the pin no longer prevents the light blocking element from obscuring the aperture or image sensor. However, the distal portion of the rod is positioned where the pin had previously been positioned and so maintains the light blocking element in the non-blocking position. When the user removes the cable from the connector, the connector moves back to or towards the first position. The rod moves back in turn, and so no longer prevents movement of the light blocking element. This allows the light blocking element to move to the blocking position. Even if a cable is again inserted into the connector, the light blocking element will remain in the blocking position, thereby preventing use of the endoscope for viewing purposes. The light blocking element can be sized to block all or only a portion of the aperture, a lens's face, or the optical sensor.

In an example embodiment, a user removes an electrical and/or optical cable from the endoscope connector, the pressure causes the connector to move backwards (e.g., slightly backwards) against the force exerted by the spring to a second position. As the connector moves forward, a rod, bar, cam, or other device in contact with the connector moves, which in turn pulls the pin into a position such that the pin no longer prevents the light blocking element from obscuring the aperture or image sensor. This allows the light blocking element to move to the blocking position. Even if a cable is again inserted into the connector, the light blocking element will remain in the blocking position, thereby preventing use of the endoscope for viewing purposes. The light blocking element can be sized to block all or only a portion of the aperture, a lens's face, or the optical sensor.

Other techniques for inhibiting reuse may be used. For example, the endoscope may contain a bladder filed with a liquid or other substance that significantly hinders optical transmission (e.g., is opaque or translucent, such an ink or dye). Upon detecting that the endoscope has been used a pre-specified number of times (e.g., once or other specified number of times) or in response to detecting that someone is attempting to use the endoscope more than the pre-specified number of times (e.g., a second time), the bladder is punctured or opened to release the substance. The bladder is positioned so the substance will flow (directly or via a guide, such as a tube or channel) onto an optical surface or to otherwise obscure an optical surface (e.g., the endoscope aperture, a lens, the image sensor, etc.). The bladder can be punctured by way of example, by a sharp pin. The pin can be moved so as to puncture the bladder by pressure being exerted on the connector, which in turn presses the pin (directly or via a translation mechanism) into the bladder. For example, the pin can be moved as similarly described above with respect to moving a pin.

By way of further example, the endoscope optionally includes a controller (e.g., a processor or state machine), that detects how many times the endoscope light source has been turned on. This can be performed via a current or voltage sensor coupled to the controller that detects when a certain voltage has been placed across the light source when a certain current is present, or when an on/off switch has been activated. By way of further example, a light sensor coupled to the controller can be used to detect when the light source has been illuminated. The controller reads and stores such use indication in memory. Optionally, a time threshold value is stored in memory, wherein the endoscope has to be “on” for at least the threshold time in order for the “on” state to be considered a use. Thus, the threshold can be used to ensure that a quick activation of the endoscope (e.g., to determine that it works) is not inadvertently considered a real use.

Once the pre-specified limited number of uses has been detected or exceeded, the controller can activate a device that inhibits further use of the endoscope. For example, the controller can open, via an electrical control signal, a mechanical or solid state relay that connects the light source to a power source to thereby prevent further illumination by the light source. Similarly, the controller can activate a relay or motor (e.g., a stepper motor) to move a light obscuring element into a light obscuring position. By way of still further example, the controller can activate a relay or motor (e.g., a stepper motor) to move a pin to puncture a bladder or open a bladder as similarly discussed above. By way of further example, the controller can prevent power and/or signals from being provided to or received by one or more elements (e.g., the light source or sensor). By way of still further example, the controller can activate a relay or motor (e.g., a stepper motor) to position an electrical insulator (e.g., a plastic or ceramic material) between two electrical contacts in a current path of the light source, thereby preventing power from being applied to the light source, or in the case of a light source that has multiple light emitting devices, from some or all of the light emitting devices.

By way of still further example, the controller can control an optical light obscuring element such as an electro-chromatic layer, LCD, or other electrically controllable optical light transmission element overlaying/underlying the endoscope aperture, a lens in the light path, and/or the image sensor. After the pre-specified number of uses, the controller causes the optical light obscuring element to transition from a substantially transparent condition to an opaque condition so as to block light (or a significant portion thereof so as to render the endoscope unsuitable for further use), wherein even if the endoscope is powered off and then powered on again the optical light obscuring element will remain in the obscuring state.

In another embodiment, a fuse (e.g., in the form of a metal wire or strip or a solid state fuse, such as diode), is used that will blow or open after a predetermined number of uses (e.g., one use or other predetermined number of uses), wherein once the fuse in blown or open certain electrical portions of the endoscope, such as the light emitting device and/or the sensor, will no longer be powered or operational. The blowing of the fuse may be under control of the controller.

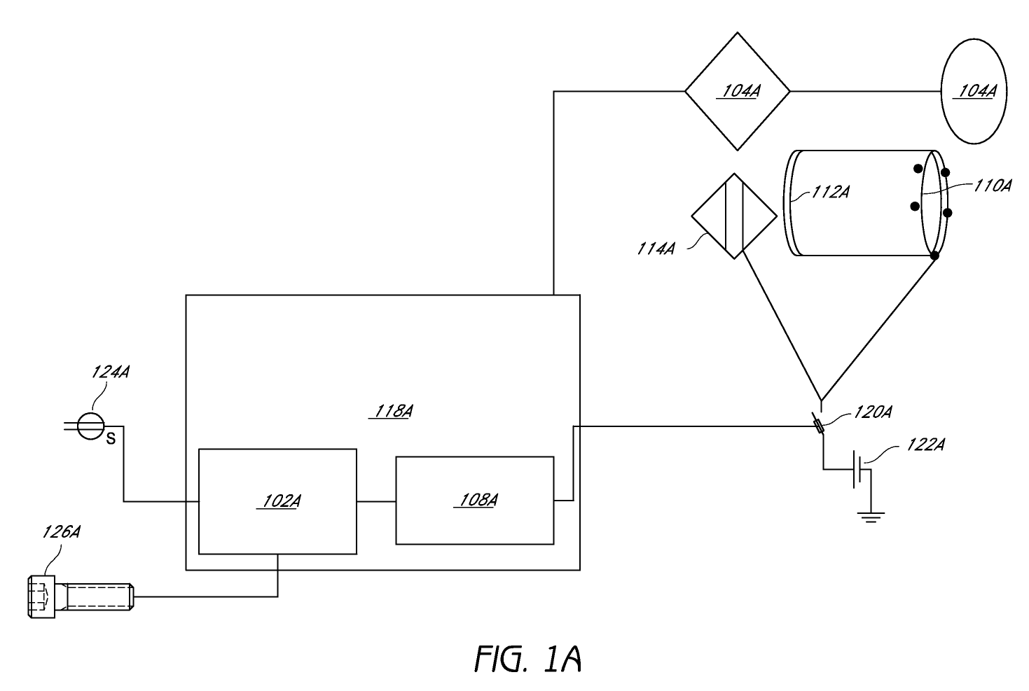

FIG. 1A illustrates an example endoscope 100A, including a disabling system 118A. Other embodiments may include fewer or additional components than those described below. The endoscope 100A includes optics component 112A configured to form images of the illuminated objects and a sensor 114A configured to detect and capture images formed by the optics component 112A. Such a sensor 114A can be, for example, a segmented detector such as a charge-coupled-device (CCD) or a complementary-metal-oxide-semiconductor (CMOS) detector. Optionally, the sensor 114A is not included in the body of the endoscope 100A, but is instead located in a separate housing that is coupled to the endoscope 100A via an optical cable.

A usage detecting device 102A detects when the endoscope has been used via one or more of the techniques described herein. For example, the usage detecting device 102A may be configured to receive an indication when a switch 124A has been turned to the ON and/or OFF positions. Optionally, in addition or instead, the usage detecting device 102A may be configured to receive an indication when a cable has been plugged into and/or removed from a cable socket 126A. Optionally, the usage detecting device 102A includes a mechanical and/or solid state memory that stores the number of times the endoscope has been used. Optionally, the usage detecting device 102A includes a mechanical and/or solid state memory that stores a predetermined number indicating the times the endoscope may be used before the endoscope is disabled.

An optics obscuring mechanism 104A is coupled to the usage detecting device 102A, using one or more of the techniques described herein. When the usage detecting device 102A detects the end of a usage event and/or the beginning of a usage event beyond the number of permitted uses, the usage detecting device 102A inhibits further use of the endoscope (e.g., via a mechanical light blocking element, an optical element with variable light transmission properties, a device that inhibits power from being applied to the light source, etc.). For example, the usage detecting device 102A at least partly causes an optional optics obscuring mechanism 104A to obscure, in whole or in part, one or more optical elements and/or detectors (e.g., the endoscope aperture, one or more lens' faces, one or more image sensors, etc.).

Optionally, in addition to or instead of the optics obscuring mechanism 104A, a light source power inhibition mechanism 108 is provided. An illumination source component 110A is included to provide illumination to a region of interest so as to allow imaging of one or more objects in the region. For the purpose of description, “illumination” is sometimes referred to as “light.” Also for the purpose of description, illumination and/or light can include visible light as commonly understood, as well as wavelength ranges typically associated with ultra-violet and/or infrared radiation. The illumination source component 110A can include one or a plurality of light emitting devices (e.g., LEDs). Non-limiting examples of the illumination source component 110A are described herein in greater detail. The light source power inhibition mechanism 108A can inhibit the light source 110A from illuminating or from adequately illuminating using one or more of the techniques described herein (e.g., using a mechanical or solid state relay or a fuse 120A).

Optionally, the endoscope 100A can include a battery 122A to power the endoscope 100A. Optionally, the endoscope 100A can be powered from a remote, separately housed power supply via a cable, as similarly described below with respect to FIG. 1.

The endoscope 100A optionally includes a visual, tactile, and/or audible indicator that indicates whether the endoscope has been disabled. For example, one or more of the components discussed above might rotate a disc having a hole there through to selectively expose a green dot (indicating that the endoscope 100A has not been disabled) or a red dot (indicating that the endoscope 100A has been disabled). Similarly, a segment or dot matrix (under control of one or more of the components discussed above) may optionally be used that indicates, via text and/or an icon, whether the endoscope 100A is functional or has been disabled (e.g., permanently disabled, such that the endoscope cannot be used without opening the endoscope body).

Certain embodiments optionally do not require that a sterilization or cleaning process be used to disable the endoscope 100A and do not require a chemical change to disable the endoscope 100A. Optionally, certain embodiments do not utilize one or more of the following techniques to disable the endoscope 100A (although other example embodiments may use one or more, two or more, three or more, four or more, five or more, or all six of the following techniques):

obscuring an optical element (e.g., optics component 112A),

moving an optical element (e.g., optics component 112A, a rod lens, etc.),

inhibiting use of a light emission device (e.g., illumination source component 110A, which may include an LED),

obscuring an image sensor (e.g., sensor 114A),

disabling a power source (e.g., a connect to battery 122A),

a chemical reaction.

For example, the endoscope 100A can be utilized with respect to one or more of the embodiments described below.

FIG. 1B illustrates a first example endoscope use inhibition process. In this example, usage determination is performed upon an initiation of use. At state 102B, a user takes an action with respect to using the endoscope. For example, the user may plug into or otherwise couple a power and/or an optical cable to a receiving socket on the endoscope. By way of further example, the user may apply power to the endoscope or remove a cap covering the socket or the light aperture. At state 104B, the usage detecting device detects the usage event. For example, the usage detecting device may detect a cable insertion by the pressure exerted on the socket, which causes the socket to move in response. By way of further example, the usage detecting device may detect an application of power by sensing a resulting voltage and/or current. By way of still further example, the usage detecting device may detect the removal of a cap from the endoscope (e.g., where the cap includes a magnet whose motion is sensed by the usage detecting device).

At state 106B, a determination is made as to whether the usage would exceed a maximum allowable usage of the endoscope. For example, the determination can be made mechanically, via a processor, via an electronic state machine device that counts uses, or otherwise.

If the usage would exceed a maximum allowable usage of the endoscope, the process proceeds to state 108B. At state 108B, the disabling system disables the endoscope. For example, the disabling mechanism may degrade the optical performance to an undesirable level, mechanically, electrically and/or optically.

By way of illustration, as similarly discussed above, the disabling mechanism can prevent the light source from illuminating or from illuminating with adequate brightness (e.g., by preventing power from being applied to the light source or reducing the amount of power supplied to the light source) and/or inhibit light from being emitted from the endoscope through an aperture and/or external light (e.g., reflected from internal portions of a patient's body) from being received by the endoscope via the aperture and/or the endoscope's optical sensor (e.g., by obscuring some or all of the endoscope aperture, one or more lenses or prisms, and/or the optical sensor).

If the usage would not exceed a maximum allowable usage of the endoscope, the process proceeds to state 110B, and the disabling mechanism permits/enables the endoscope to be fully utilized (e.g., allows the light source to be turned on and does not obscure optical elements or the sensor).

FIG. 1C illustrates another example endoscope use inhibition process. In this example, usage determination is performed upon cessation of use of the endoscope. At state 102C, a user takes an action with respect to ceasing use of the endoscope. For example, the user may unplug into or otherwise uncouple a power and/or an optical cable from a receiving socket on the endoscope. By way of further example, the user may turn off power to the endoscope or position a cap so as to cover the socket or the light aperture. At state 104C, the usage detecting device detects the cessation of use event. For example, the usage detecting device may detect a cable removal by the pressure exerted on the socket when the cable is pulled from the socket, which causes the socket to move in response. By way of still further example, the usage detecting device may detect the placement of a cap on the endoscope (e.g., where the cap includes a magnet whose motion is senses by the usage detecting device).

At state 106C, a determination is made as to whether the endoscope has been used the maximum allowable number of times. For example, the determination can be made mechanically, via a processor, via an electronic state machine device that counts uses, or otherwise.

If the endoscope has been used the maximum allowable number of times, the process proceeds to state 108C. At state 108C, the disabling system disables the endoscope. For example, the disabling mechanism may degrade the optical performance to an undesirable level, mechanically, electrically and/or optically.

By way of illustration, as similarly discussed above, the disabling mechanism can prevent the light source from illuminating or from illuminating with adequate brightness (e.g., by preventing power from being applied to the light source or reducing the amount of power supplied to the light source) and/or inhibit light from being emitted from the endoscope through an aperture and/or external light (e.g., reflected from internal portions of a patient's body) from being received by the endoscope via the aperture and/or the endoscope's optical sensor (e.g., by obscuring some or all of the endoscope aperture, one or more lenses or prisms, and/or the optical sensor).

If the usage does not exceed the maximum allowable usage of the endoscope, the process proceeds to state 110C, and the disabling mechanism permits/enables the endoscope to be fully utilized (e.g., allows the light source to be turned on and does not obscure optical elements or the sensor) the next time it is turned on.

The example endoscope, devices, and/or systems discussed above can be utilized with one or more of the embodiments discussed below.

FIG. 1D illustrates one system 100 for producing images such as electronic, e.g., video or digital, images of features inside, for example, body parts. The system 100 includes an endoscope structure 110 (e.g., such as all of or portions of the endoscope 100A described above, including the disabling system 118A) coupled to an imaging and control apparatus 114 through a cable 112. The imaging and control apparatus 114 includes an optical sensor 116, a processor 118, a display 120, a power supply 122, and a power control 124. Optionally, the sensor 116, processor 118, power supply 122, and power control 124 are positioned within the body of the endoscope structure 110.

The endoscope structure 110 comprises an elongated member that is inserted into a portion of a body such as a human body. This endoscope structure 110 includes a distal end portion 126 and a proximal end portion 128. One or more solid state emitters (not shown) are preferably disposed at the distal end portion 126. The solid state emitters each include an electrical input and have an optical output. The solid state emitters may comprise, for example, light emitting diodes (LEDs). Preferably, these solid state emitters are bright and small. In some embodiments, for example, these solid state emitters radiate over 10 lumens. These LED may be less than a millimeter and in some embodiments may be about 0.5 millimeters. The large brightness and small size of these emitters enables such endoscopes to have a smaller cross-section than conventional endoscopes that rely on large optical fiber bundles to provide illumination. Reduced size offers the advantage that the endoscope is less intrusive and causes less damage and trauma to the body. A plurality of such small solid state emitters may be disposed at the distal end of the endoscope structure 110. In certain embodiments 2, 3, 4, 5, 6, 7, 8, or more emitters are employed. In some embodiments, these emitters emit white light although emitters need not be white light emitters. Colored emitters and emitters that radiate in narrow wavelength ranges may be employed as well. For example, images may be formed by optical sensors 116 that are sensitive to the particular wavelength region used for illumination. In certain embodiments, a specific wavelength illumination may be employed for fluorescence applications.

The solid state emitters radiate light and illuminate a portion of a body cavity. Accordingly, the distal end 126 of the endoscope structure 110 includes an aperture (not shown) for collecting light reflected or scattered from the illuminated portion of the body cavity. The light collected through the aperture is transferred along an optical path (not shown) from the distal end 126 of the endoscope structure 110 to the proximal end 128. Preferably, features in the illuminated portion of the cavity are imaged and the image is relayed along the optical path so as to form an image of a portion of the body cavity at the proximal end 128.

Accordingly, the light and image are transferred from the proximal end 128 of the endoscope structure 110 through the cable 112 to the imaging and control apparatus 114. Accordingly, the cable preferably comprises a system of relay lens or a coherent fiber bundle. The cable preferably transfers the image to the optical sensor 116 in the imaging apparatus 114. The optical sensor 116, which may comprise a detector array such as a CCD or CMOS sensor array, has a light sensitive optical input that receives the light from the cable 112. The optical sensor 116 preferably further comprises an electrical signal output for outputting an electrical signal corresponding to the image of the illuminated portion of the body cavity. The electrical signal from the optical sensor 116 is transmitted to a processor 118 and onto a on the display device 120 such as a video screen or computer monitor. Although not shown, alternative embodiments may include transmitting the electrical signal from the optical sensor 116 directly to the display device 120, for example, when the optical sensor 116 performs the processing.

As discussed above, in certain embodiments the cable 112 comprises a fiber optic such as a coherent fiber optic bundle. The cable 112 also preferably includes electrical power lines (not shown), such as thin electrical leads or wires, that provide electrical power to the solid state emitters disposed at the distal end 126 of the endoscope 110. The electrical power lines are electrically coupled to the power supply 122. This power supply 122 may, for example, provide 12 or 24 volts and 20 milliamps to 1.5 Amp of current, however, voltages and currents outside these ranges are possible. The power supply 122 may be controlled by the power controller 124. The power controller 124 may, for example, enable the current supplied to the solid state emitters at the distal end 126 of the endoscope structure 110 to be adjusted. Accordingly, the brightness or intensity of the light emitted from the solid state emitters can be adjusted. In one embodiment, the power control comprises a rheostat.

Although the cable 112 is included in the endoscope system 100 shown in FIG. 1D, this cable is not required. In other embodiment, this cable 112 may be excluded. For example, the optical sensor 116 may be disposed at the proximal end portion 128 of the endoscope structure 110. In such designs, electrical cable may be connected to the endoscope structure 110 to power the one or more solid state emitters at the distal end portion 126.

In certain embodiments, the endoscope structure 110 is disposable. Various design features discussed more fully below may reduce the cost of the endoscope structures 110 such that the endoscope structure need not be reused over and over but may be discarded after use. In some embodiments, the endoscope structure 110 may plug into the cable 112 and thus may be detached and disposed of and replaced for the next procedure.

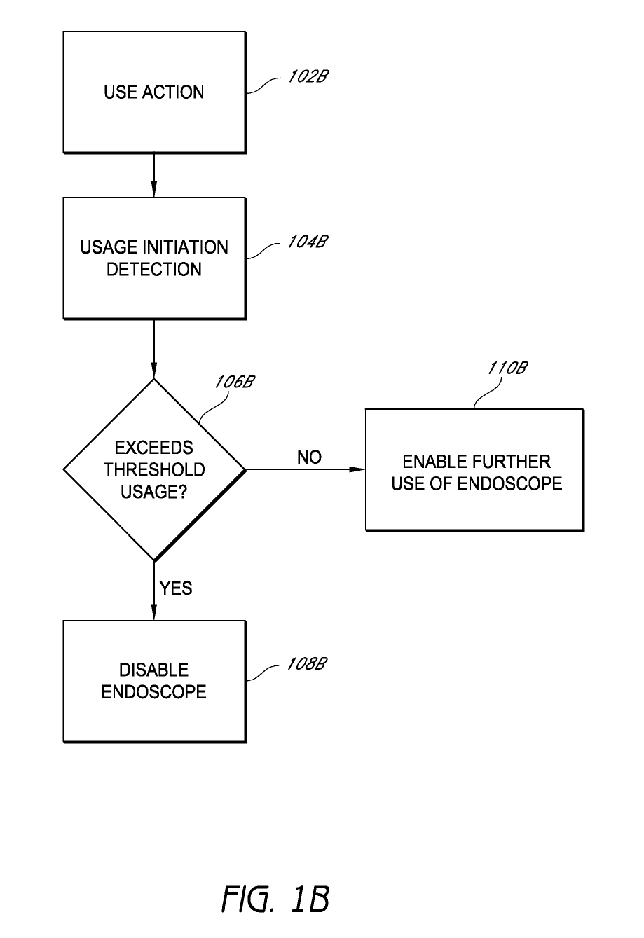

FIG. 2 illustrates a system 200 that offers increased ease of use. The system 200 includes an endoscope structure 220, a receiver 222, a processor 224, and a display device 226. The endoscope shown in FIG. 2, however, is a battery operated, hand-held instrument which is configured to produce images of internal regions of a body as described above. The endoscope structure 220 (e.g., which may be in the form of endoscope 100A described above, including the disabling system 118A) shown includes a distal end 230 and a proximal end 232 and one or more solid state emitters (not shown) at the distal end that emit light to illuminate internal regions of the body. The distal end 230 of the endoscope structure 220 further includes an aperture (not shown) for collecting light emitted from the solid state emitters and reflected off of the internal regions of the body. An optical path (not shown) extends from the distal end 230 of the endoscope structure 220 to a proximal end 232.

At the proximal end 232 of the endoscope structure 220 is an optical sensor 234, a transmitter 236, a battery 238, and a control device 240. The optical sensor 234 is disposed to receive collected light and more particularly, an image of a portion of the body, and to provide an electrical signal output. At the proximal end 232, the light collected at the distal end 230 forms an image on the optical sensor 234 which produces an electrical output corresponding to the image of the illuminated internal region of the body. The electrical signal is supplied to the transmitter 236, which transmits the signal to the receiver 222. The transmitter 236 and the receiver 222 are preferably wireless. In various embodiments, the transmitter 236 comprises an RF transmitter and the receiver 222 comprises an RF receiver. The receiver 222 provides the received signal to the processor 224 that feeds signals to the display device 226. In some embodiments, the processor 224 may format the received signal so that the image of the illuminated internal region of the body can be displayed. This processor 224 may also provide additional image processing. In alternative embodiments, the optical sensor 234 provides the necessary formatting and processing and the received signal is transferred directly from the receiver 222 to the display device 226. Other distributions of functions between electronics in the optical sensor 234 and processor 224 are possible.

The battery 238 is electrically coupled to the transmitter 236, the optical sensor 234 and to the solid state emitters disposed at the distal end 230 of the endoscope structure 220. The control device 240 may be configured to allow a user of the endoscope to control the amount of current supplied by the battery 238 to the solid state emitters disposed at the distal end 230 of the endoscope structure 220. In an embodiment, the control device 240 is also configured to allow the user to selectively apply or remove a power signal from the battery 238 to the transmitter 236 and solid state emitters. This controller device 240 may comprise, for example, a rheostat or potentiometer, or digital switch, in certain embodiments. The control device may comprise an integrated circuit chip, such as a microprocessor, in certain embodiments.

The optical sensor 234, transmitter 236, and battery 238 disposed at the proximal end 232 of the endoscope structure 220 allows the endoscope structure to be a self-contained instrument that is easily maneuverable and readily mobile. The endoscope structure 220 does not need to be attached with wires or cables to provide power or to carry an image or signal to processing and display instruments. The user therefore has increased freedom to manipulate the endoscope structure and is not tethered to a console or power supply that would otherwise restrict the range of movement during a procedure. As described above, in various embodiments, the endoscope structure 220 is disposable. In certain embodiments, the endoscope structure 220, including the solid state emitters, is disposable and is detachable from the optical sensor 234, transmitter 236, battery 238, and control device 240, which are reusable. Various design features help reduce the cost of the endoscope structure 110 and enable disposal and replacement to be a competitive alternative to reuse.

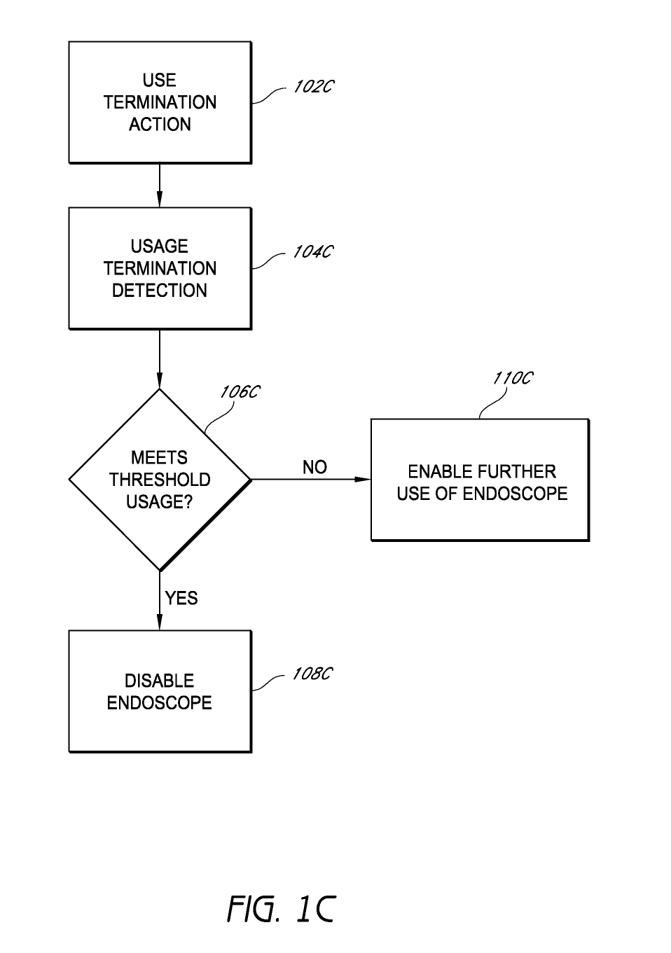

FIG. 3 illustrates an exploded perspective view of a longitudinal member 300 comprising an endoscope structure. The longitudinal member 300 has a distal end 320 and a proximal end 322. The longitudinal member 300 has a hollow inner cavity region 324 which provides an optical path from the distal end 320 to the proximal end 322.

A plurality of solid state emitters 326 (five shown) are disposed at the distal end 320 of the longitudinal member 300. In various embodiments, the solid state emitters 326 each comprise an LED. The solid state emitters are configured to emit light into the body.

At the distal end 320, the longitudinal member 300 includes a front lens holder 328 having a front surface 332 with seats to receive the solid state emitters 326. The front lens holder 328 also includes a channel therethrough that comprises a portion of the inner cavity region 324 of the longitudinal member 300. Front and rear apertures in the front lens holder 328 provide access to the channel and a path through the lens holder 328. Illumination reflected from portions of the body proceeds through this channel along this optical path. Preferably, the front lens holder 328 is configured to hold a front lens 330 that collects reflected light from the solid state emitters 326 into the inner cavity region 324 of the front lens holder 328. In certain preferred embodiments, the front surface 332 is angled so that light can be collected at the distal end 320 from an oblique direction with respect to the longitudinal member 300. For example, the longitudinal member 300 may be used to observe an inner side wall of a vessel such as a vein or artery by inserting the longitudinal member 300 longitudinally into the vessel and rotating the longitudinal member 300 such that the tilted front surface 332 is directed towards a portion of the inner side wall of the vessel desired to be imaged.

The longitudinal member 300 further includes a cradle 340 that is attachable to the front lens holder 328. The cradle 340 is configured to be a support structure for at least one optical element in the optical path from the distal end 320 of the longitudinal member 300 to the proximal end 322 of the longitudinal member 300. In various embodiments, the cradle 340 is configured to support and align multiple lens elements 342 (five shown). The lens elements 342 may comprise, for example, rod lenses. The cradle 340 is an elongated support structure comprising a hollow cylindrical tube with portions of the tube removed to form slots 344 (five shown). In various embodiments, the slots 344 are sized, configured, and positioned to receive the lens elements 342 and to align the lens elements 342 automatically along the optical path in the inner cavity region 324. Moreover, the slots 344 are preferably spaced apart to provide the appropriate spacing of the lens 342 along a longitudinal direction and optical axis as defined by the lens prescription.

The longitudinal member 300 further comprises an outer tube 350. The outer tube 350 includes an inner region 352 and an outer region 354. With the lens elements 342 disposed in the slots 344 of the cradle 340, the cradle 340 can be slid into the inner region 352 of the outer tube 350. The outer tube 350 may shield and protect the cradle 340 and lens elements 342.

In certain embodiments, the outer region 354 of the outer tube 350 comprises a heat conducting material such as aluminum, stainless steel, or the like. In such an embodiment, the outer tube 350 may conduct heat generated by the solid state emitters 326 away from the distal end 320 of the longitudinal member 300. In other embodiments, other portions of the outer tube 350, the cradle 340, and/or lens holder 328 may comprise thermally conducting material. Conductive material may be deposited on the outer tube 350, the cradle 340 and/or the lens holder 328 in certain embodiments. For example, these components may comprise ceramic or plastic with portions having metallization formed thereon by, for example, electroplating or electrochemically deposition. In certain embodiments, the outer tube 350 comprises stainless steel and a portion of this outer tube 350 is electroplated with aluminum for heat conduction and/or electrical connection. Other designs are possible.

Although not shown, a diffuser or a plurality of diffusers may be disposed in front of the solid state emitters 326. The diffuser or plurality of diffusers are configured to disperse the light from the solid state emitters 326.

In operation, at least the distal end 320 of the longitudinal member 300 is inserted into a body cavity. An electrical power signal is provided to the solid state emitters 326 by thin electrical wires (not shown) or electrical traces (not shown) that may be disposed along a surface of the cradle 340 and front lens holder 328. The electrical power signal causes the solid state emitters 326 to emit light having an intensity proportional to the electrical power signal. In the case where the longitudinal member 300 comprise conducting material such as metal, the conducting longitudinal member 300 may operated as an electrical path for providing power or grounding to the emitters 326.

The light is reflected off an object within the body cavity or the inner walls of the body cavity. A portion of the reflected light is collected into the inner cavity region 324 of the front lens holder 328 through an aperture (not shown) in the front surface 332. As discussed above, the light may be collected by a front lens 330. The light is then directed through the plurality of lens elements 342 disposed in the cradle 340. Thus, the light propagates from the distal end 320 of the longitudinal member 300 to the proximal end 322 of the longitudinal member 300. The lens elements 342 are preferably positioned and aligned by the cradle so as to relay an image of the illuminated object or inner wall.

The solid state emitters 326 generate heat as they emit light. The heat is preferably conducted away from the distal end 320 of the longitudinal member 300 by the heat conducting surface 354 of the outer tube 350. In other embodiments, other portions of the outer tube, the cradle 340 and/or lens holder 328 may comprise thermally conductive material or layers so as to transfer heat produced by the emitters 326. Increased thermal conduction permit the emitters 326 to be driven with more power so as to emit more light. In some embodiments, the LEDs are driven with a current of up to 40 or 60 milliamps or more.

Preferably, the longitudinal member 300 has a small cross-section for example less than 3 or 4 millimeters across in some embodiments. The small size of the emitters facilitates such small cross-sections. As described above, the small cross-section reduces trauma and damage to the body in which the endoscope is inserted.

In various embodiments, the longitudinal member 300 is disposable. The lenses 342 may comprise compression molded glass, which can be manufactured relatively inexpensively such that the longitudinal member 340 together with the emitters 326 and the lens may be disposed of after a single use and remain cost-effective in comparison with conventional endoscope designs. In certain embodiments, the longitudinal member is sterilizable.

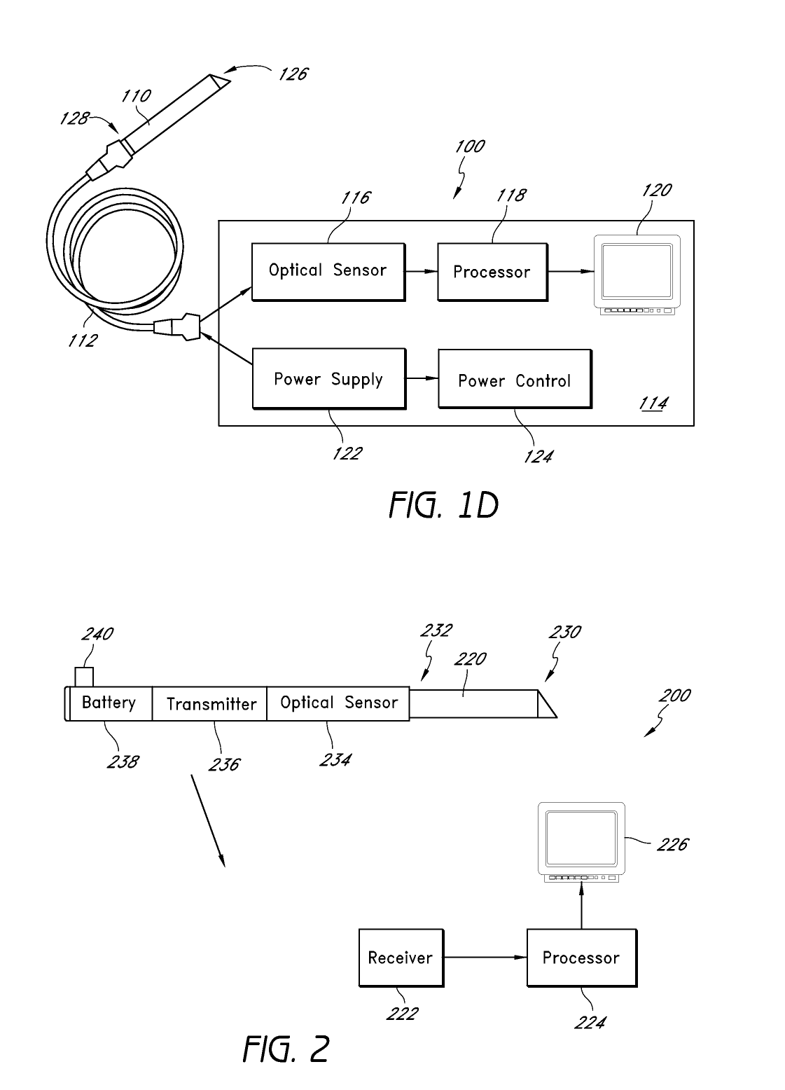

FIG. 4 is a rear perspective view of an exemplary front lens holder 400 for use with a longitudinal member of an endoscope, such as the longitudinal member 300 shown in FIG. 3. The front lens holder 400 comprises a front surface 402, a rear surface 404, and an inner cavity region 406. The front surface 402 and the rear surface 404 each comprise an aperture to the inner cavity region 406. For illustrative purposes, FIG. 4 shows an optical path 410 entering the aperture on the front surface 402, passing through the inner cavity region 406 and out the aperture of the rear surface 404.

The front surface 402 is tilted with respect to the rear surface 404 of the front lens holder 400. The tilted front surface 402 allows the front lens holder 400 to collect light reflected from of objects located to the side of an endoscope. In exemplary embodiments, the front surface 402 is tilted between about 30° and 70° with respect to the rear surface 404. In certain embodiments, for example, this tilt may be about 45°. However, it should be noted that the tilt of the front surface 402 can be selected to provide the user of the endoscope with the ability to view objects located to the side of the endoscope according to any number of angle ranges, including but not limited to a flat surface parallel to the rear surface 404. In some of these embodiments, solid state emitters (not shown) located on the front surface 402 may be angled, for example, so as to emit light at an angle to illuminate objects to the side of the endoscope. The lens (not shown) in the lens holder 400 may also be tilted to collect light reflected or scattered from the sidewalls of the body cavity.

The front lens holder 400 is configured to redirect the light entering the front lens holder 400 through the aperture in the front surface 402 to exit the front lens holder 400 through the aperture in the rear surface 404 so as to convey an image of an object along an optical path through the endoscope. In certain embodiments, the light entering the front lens holder 400 is redirected using an optical element such as a prism (not shown) comprising one or more reflective surfaces. In various preferred embodiments, however, the light entering the front lens holder 400 is redirected using a first reflective surface 420 and a second reflective surface 422. Preferably, the first and second reflective surfaces 420, 422 do not comprise glass. These reflective surfaces 420, 422 may comprise a reflective layer such as metallization formed on a surface of the lens holder 400.

FIG. 4 illustrates the first reflective surface 420 and the second reflective surface 422 walls defining the inner cavity region 406. The first reflective surface 420 and the second reflective surface 422 are angled such that the optical path 410 of the light entering the cavity region 406 approximately perpendicular to the front surface 402 will be redirected so as to exit the cavity region 406 approximately perpendicular to the rear surface 404. Thus, for example, light entering the longitudinal member 300 shown in FIG. 3 will be redirected and conveyed through the inner cavity region 324 from the distal end 320 to the proximal end 322 through the plurality of rod lenses 342.

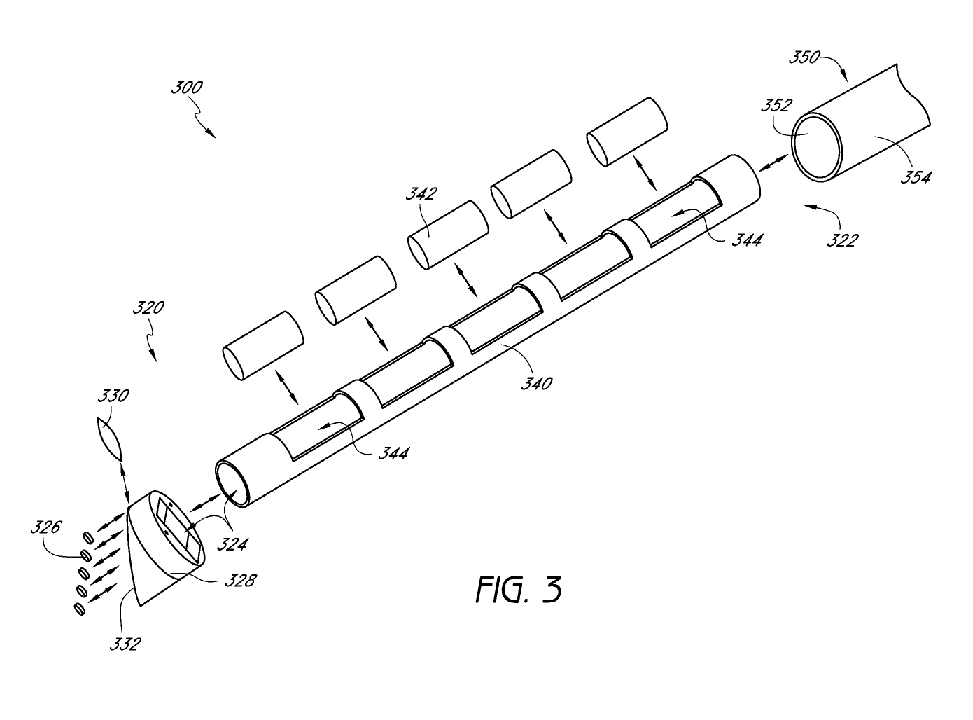

To illustrate the concept of redirecting light through the front lens holder 400, FIG. 5 shows a schematic diagram of an optical path 508 through a front surface 510 tilted at an angle with respect to a rear surface 512. The optical path 508 passes approximately perpendicular through the front surface 510 and intersects with a first reflective surface 514 positioned and angled so as to redirect the optical path 508 to a second reflective surface 516. The second reflective surface 516 is positioned and angled so as to redirect the optical path 508 approximately perpendicularly through the rear surface 512. In other embodiments, the front surface 510 and rear surface 512 may not be perpendicular to this optical path 508, however, preferably the first and second reflective surfaces 514, 516 are oriented to direct the optical path through the length of the elongated member.

Referring again to FIG. 4, the first and second reflective surfaces 420, 422 are substantially specularly reflective. The first and second reflective surfaces 420, 422 may, for example, be smooth, planar surfaces. The front lens holder 400 may be formed from materials that can be molded or machined. In various embodiments, the front lens holder 400 is formed of a material selected from the group comprising plastic, ceramic, or metal such as nickel or the like. In certain preferred embodiments, the first and second reflective surfaces 420, 422 are polished until they are substantially smooth. For example, the first and second reflective surfaces 420, 422 may be polished down to average roughness of approximately eight Angstroms. After polishing, the first and second surfaces may be metalized with a substantially reflective material, such as nickel, chrome or the like. Other reflective layers may be employed as well. In certain embodiments, the substantially reflective material is electroplated or electrochemically deposited onto the polished surfaces. For example, in various exemplary embodiments, the lens holder comprises molded or machined plastic or ceramic that is electroplated to form reflective metal layers. Nickel electroforming, for example, may be employed to create the first and/or second reflective surfaces 420, 422. Such processes are well-developed and relatively inexpensive and can be readily implemented in manufacturing processes.

Forming reflective surfaces on the inner walls of the lens holder offers several advantages. Integrating the reflective surfaces into the lens holder reduces the number of elements that need to be optically aligned. For example, once the reflective surfaces have been formed on the interior walls of the lens holder, precise alignment may be achieved by simply inserting or “snapping” the lens holder 400 in place on the longitudinal member 300. In contrast, microscopes are employed to align tiny prisms in conventional designs. These micro-prisms are also substantially more expensive. For example, injection molding the lens holder 400, polishing inner surfaces on the lens holder, and performing Ni electroforming or chrome electroplating may be relatively less expensive in comparison to polishing tiny glass micro-prisms. The reduced cost yielded by such designs may permit the endoscope to be disposable.

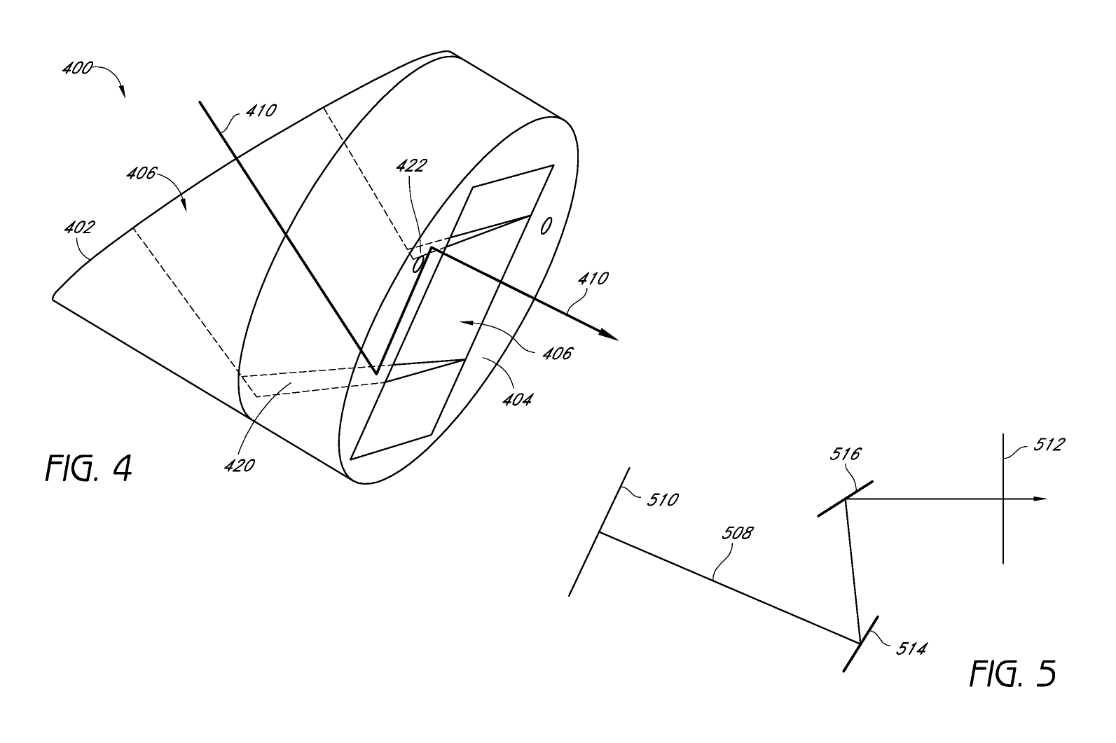

FIG. 6 provides another view of a front lens holder 600 for use with a longitudinal member of an endoscope, such as the longitudinal member 300 shown in FIG. 3. FIG. 6 is a partial front perspective view of the front lens holder 600. The front lens holder 600 comprises a front surface 610 and a rear surface 612. A hollow interior region 614 extends from an aperture in the front surface 610 to an aperture in the rear surface 612. In various embodiments, the front lens holder 600 includes a lens seat 616 configured to hold a lens (not shown) which covers the aperture in the front surface 610. The specifications of the lens, e.g., power, numerical aperture, etc., are preferably selected to direct light into the front lens holder 600. Alternatively, the aperture in the front surface 610 may be covered with a window or material (not shown) that is transparent to selected wavelengths of light. A lens may be disposed in the inner region 614 of the lens holder 600 or may be exterior to the lens holder in some embodiments. The hollow interior region 614 may be hermetically sealed and may be filled with a gas or liquid. Alternatively, the hollow interior region 614 may be a vacuum.

The front surface 610 of the front lens holder 600 includes a plurality of seats 622 (eight shown) configured to hold solid state emitters (not shown), such as LEDs. The seats 622 are positioned around the aperture in the front surface 610. The seats 622 are positioned such that light emitted from their respective locations will be reflected from an object back through the aperture in the front surface 610. In various embodiments, the seats are arranged to provide substantially uniform illumination.

The front surface 610 also includes a path 624 for electrical power. In an embodiment, the path 624 is shaped to hold thin electrical wires connecting the solid state emitters to an electrical power source. Alternatively, the path 624 comprises a conductive trace for providing power to the solid state emitters. The path 624 may be connected to one or more through-holes 626 (two shown) to electrically couple power from a power source (not shown).

As described above, the front lens holder 600 may be formed, for example, by molding, machining, or other manufacturing processes. The lens holder may comprise two or more separable pieces that are fit together. Such designs may facilitate manufacture such as polishing the inner surfaces to form reflective portions of the interior sidewalls. In various embodiments, the front lens holder 600 is disposable and/or sterilizable.

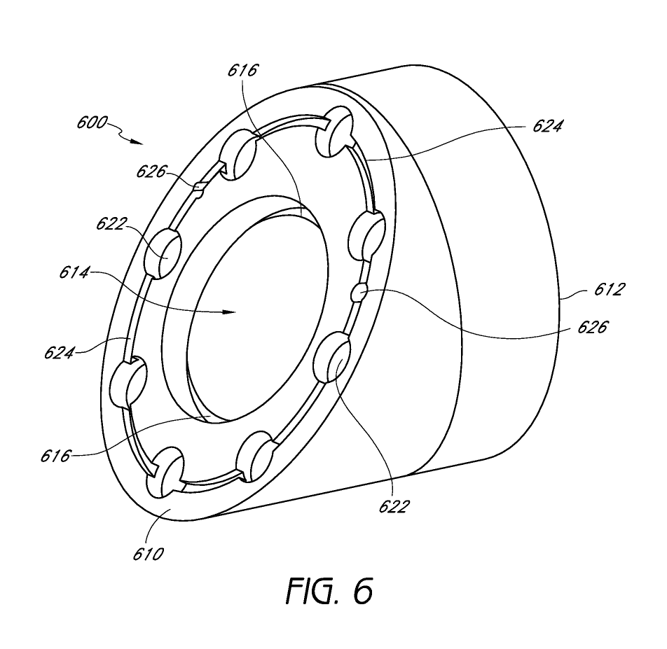

FIG. 7 is a perspective view of an elongated support structure 700, which can be used as a cradle, such as the cradle 340 shown in FIG. 3. The elongated support structure 700 comprises a hollow tube 710 having a plurality of slots 712 (five shown) each configured to hold a lens such as a rod lens (not shown) or other optical element. The slots 712 are separated by spacer portions 714 (four shown) that are each sized and positioned so as to provide proper alignment and longitudinal separation of the rod elements for suitable relay of an image therethrough. In other words, the spacing between the slots 712 are defined by the spacer portions 714 so as to longitudinally space the rod lenses with respect to each other according to the optical design prescription.

The elongated support structure 700 may be formed, for example, by molding, machining, or other manufacturing processes. The elongated support structure 700 may comprise, for example, plastic, ceramic, or metal. In certain embodiments, one or more electrical traces or paths may be formed on a surface of the elongated support structure 700 to provide electrical power to solid state light emitters (not shown). In various embodiments, the elongated support structure 700 is sterilizable and/or disposable.

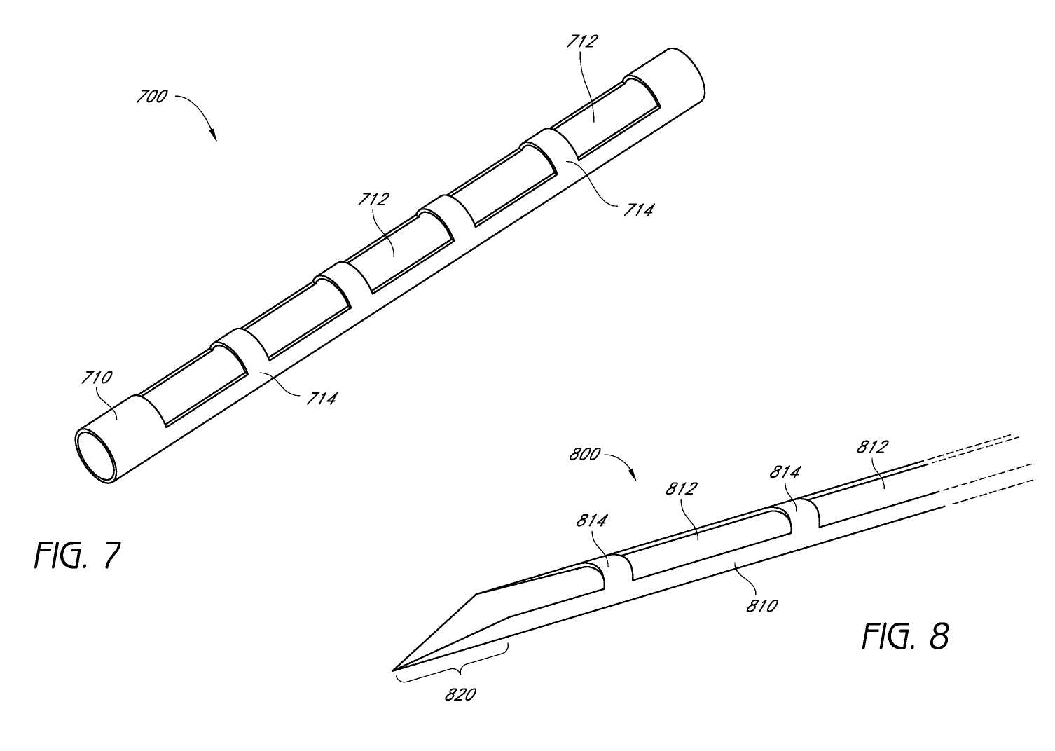

FIG. 8 is a partial perspective view of another exemplary slotted elongate support structure 800 which can be used as a cradle, such as the cradle 340 shown in FIG. 3. The slotted elongate support structure comprises a hollow tube 810 having slots 812 configured to hold lens such as rod lens (not shown) or other optical elements. The slots 812 are separated by spacing elements 814 (two shown) that are each sized and positioned so as to provide proper longitudinal separation of the rod elements for suitable propagation of an image. The slots 812 are preferably positioned to provide proper lateral positioning of the lens or other optical elements as well.

The slotted elongate support structure 800 also includes a tapered “V” shaped portion 820 that is pointed at one end. The tapered “V” shaped portion 820 is configured to facilitate the insertion of the slotted elongate support structure 800 into an outer tube, such as the outer tube 350 shown in FIG. 3. When aligning the slotted elongate support structure 800 with an outer tube, the point of the “V” shaped member 820 is preferably sufficiently small so as to be easily inserted into the outer tube. The “V” shaped member 820 also simplifies the manufacturing process by properly aligning the slotted elongate support structure 800 with an outer tube upon insertion therein.

The slotted elongate support structure 800 may have other shapes as well. In certain embodiments, for example, the slotted elongated support structure may be “V” shaped having a “V” shaped lateral cross-section over a substantial portion of its length.

The features described herein can be employed alone or in various combinations to create improved endoscopes designs. For example, endoscope structures having solid state emitters may be employed together with a lens holder that does not include a prism. Alternatively, the lens holder designs described herein can be employed with conventional illumination approaches such as use of a fiber optic bundle instead of LEDs. Similarly, the slotted elongated support structure may be employed with or without solid state emitters and with or without the lens holder having reflective interior sidewalls for directing an image through an array of lenses. A wide range of designs are possible.

Also, although FIG. 3 depicts rod lenses being disposed in the endoscope structure, in various embodiments, other types of lenses such as lenses having reduced longitudinal thickness may be employed. Rod lenses advantageously increase optical throughput by increasing the Lagrange invariant. However, a plurality of small bright solid state light emitters, such as LED's, may provide substantially illumination. The solid state emitters, together with their electrical power connections, however, do not occupy as much area across a lateral cross-section of the endoscope structure as a fiber optic bundle used for illumination in conventional endoscope designs. Accordingly, room is available for larger diameter lenses having higher numerical aperture and throughput when using tiny solid stated emitters. With increased throughput, lenses thinner than rod lens may be employed. The reduced Lagrange invariant is offset by the increase in diameter of the lenses. The throughput may be larger in some cases where thin lenses are employed instead of rod lenses. Likewise, rod lenses may or may not be employed in combination, for example, with the lens holder having internal reflecting sidewalls and/or the slotted elongate support structure. For example, in certain embodiments, the elongate support structure may have slots with reduced length to accommodate lenses other than rod lenses. In general, rod lenses are more expensive than thin lenses. Accordingly, the manufacturing cost of the endoscope can be reduced.

As described above, various combination and arrangements may be employed. Accordingly, the structures and apparatus should not be limited to those particular designs shown in FIGS. 1-8 or specifically disclosed in the description of these figures. Other embodiments are possible as well. These embodiments may include features well known in the art as well as feature not yet devised.

As described above, the process of manufacturing the endoscope devices may be simplified or improved. In certain embodiments, for instance, the lenses can be automatically positioned in the cradle so as to have suitable spacing between lenses to relay an image in the body. Such a method of forming an endoscope apparatus having proximal and distal ends may comprise, for example, providing an elongated support structure having a plurality of sites for insertion of optical elements and inserting a plurality of lenses at the sites. The elongated support structure may be inserted into a hollow outer protective shield having an open inner region. Preferably, the plurality of sites are laterally positioned and longitudinally spaced with respect to each other so as to provide an aligned optical system that relays an image from the distal end portion to the proximal end portion. Such manufacture may be implemented partially or totally robotically in certain cases. Such automated processes may reduce the cost of manufacture.

In other various embodiments, a front endpiece may be attached at the distal end portion of an endoscope assembly. The front endpiece preferably has an open inner region for receiving light to form images of portions of a body. A plurality of solid state light emitters are preferably affixed to the front endpiece to illuminate the body portions. A lens is mounted to the front endpiece to receive light from the body portions. At least one reflective surface is formed on a sidewall of the inner open region of the front endpiece to reflect light received from the body portions through the plurality of lenses.

Other manufacturing methods may include molding the front endpiece so as to include the sidewall surface on the inner open region for forming the reflective surface with a shape and orientation to produce the image. The reflective surface may be formed by metalizing the sidewall surface. In certain embodiments, the sidewall surface is polished prior to metallization.

In other embodiments, a method for manufacturing a front end of an endoscope for viewing portions of a body comprises forming a front endpiece for receiving light from the body portions so as to enable viewing of the body portions. An inner cavity region is formed in the front endpiece to allow passage of the light from the body portions and at least one substantially planar sidewall surface is formed in the inner cavity region. The method also includes metalizing the at least one substantially planar sidewall surface so as to form a substantially reflective surface that reflects the light received from the body portions. The sidewall surface may be polished prior to metallization to create a substantially smooth surface.

At least one seat is preferably formed in the front endpiece for placement of one or more solid state light emitters to illuminate the body portions. A lens seat may be formed in the front endpiece for mounting a lens to receive light from the body portions. In certain embodiments, the front endpiece is formed by molding. In some embodiments, at least a portion of the front endpiece is formed by machining.

Various combinations of manufacturing steps may be employed with more or less steps and the specific method should not be limited to the specific processes recited herein. A wide range of fabrication methods are possible.

In one or more example embodiments, the functions, methods, algorithms, and techniques described herein may be implemented in hardware, software, firmware (e.g., including code segments), or any combination thereof. If implemented in software, the functions may be stored on or transmitted over as one or more instructions or code on a computer-readable medium. Tables, data structures, formulas, and so forth may be stored on a computer-readable medium. Computer-readable media include both computer storage media and communication media including any medium that facilitates transfer of a computer program from one place to another. A storage medium may be any available medium that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

For a hardware implementation, one or more processing units at a transmitter and/or a receiver may be implemented within one or more computing devices including, but not limited to, application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a software implementation, the techniques described herein may be implemented with code segments (e.g., modules) that perform the functions described herein. The software codes may be stored in memory units and executed by processors. The memory unit may be implemented within the processor or external to the processor, in which case it can be communicatively coupled to the processor via various means as is known in the art. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Although certain embodiments and examples are discussed herein, it is understood that the inventive subject matter extends beyond the specifically disclosed embodiments and examples to other alternative embodiments and uses and to obvious modifications and equivalents thereof. Thus, it is intended that the scope of the disclosure should not be limited by the particular disclosed embodiments and examples. For example, in any method or process disclosed herein, the acts, steps, or operations making up the method/process may be performed in any suitable sequence and are not necessarily limited to any particular disclosed sequence. Also, acts, steps, or operations may be added, removed, combined, or rearranged in other method/process embodiments. In systems and devices disclosed herein, components may be added, removed, combined, and/or arranged differently than described herein.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein. Further, embodiments may include several novel features, no single one of which is solely responsible for the embodiment's desirable attributes or which is essential to practicing the systems, devices, methods, and techniques described herein.

What is claimed is:

1. An endoscope, comprising:
a lens;
a usage detector; and
a disabling device coupled to the usage detector, wherein the disabling device is configured to permanently disable the endoscope at least partly in response to:
(a) a mechanical output from the usage detector,
(b) an optical output from the usage detector,
(c) electrical output from the usage detector, or
(d) any combination of (a), (b), or (c),
wherein the usage detector is configured to detect:
(i) an initiation of use of the endoscope,
(ii) a termination of use of the endoscope,
(iii) a cable insertion,
(iv) a cable removal,
(v) a switch activation, or
(vi) any combination of (i), (ii), (iii), (iv), or (v).

2. The endoscope of claim 1, wherein the disabling device is configured to disable an LED (light emitting diode) mounted within the endoscope.

3. The endoscope of claim 1, wherein the usage detector is configured to detect an initiation of use of the endoscope.

4. The endoscope of claim 1, wherein the usage detector is configured to detect a termination of use of the endoscope.

5. The endoscope of claim 1, wherein the usage detector is configured to detect a cable insertion and/or removal.

6. The endoscope of claim 1, wherein the usage detector detects a switch activation.

7. The endoscope of claim 1, wherein the disabling device is configured to disable the endoscope by at least partly obscuring an optical element.

8. The endoscope of claim 1, wherein the disabling device is configured to disable the endoscope by inhibiting illumination of a light source.

9. The endoscope of claim 1, wherein the disabling device includes an electrically controllable optical light transmission element.

10. The endoscope of claim 1, wherein the disabling device includes an electrically controllable optical light transmission element including an electro-chromatic layer and/or a liquid crystal.

11. The endoscope of claim 1, wherein the disabling device includes a fuse.

12. The endoscope of claim 1, wherein the disabling device only permits a single use of the endoscope before disabling the endoscope.

13. The endoscope of claim 1, further comprising a light emission device, an image sensor, and/or a power source, wherein the disabling device is configured to disable the endoscope by disabling the light emission device, the image sensor, and/or the power source.

14. The endoscope of claim 1, wherein the disabling device does not rely on a sterilization process to disable the endoscope.

15. A method for disabling an endoscope, comprising:
detecting, via a usage detector device, a usage event, the usage event including one or more of:
(i) an initiation of use of the endoscope,
(ii) a termination of use of the endoscope,
(iii) a cable insertion to the endoscope,
(iv) a cable removal from the endoscope,
(v) a switch activation, or
any combination of (i), (ii), (iii), (iv), or (v); and
permanently disabling the endoscope at least partly in response to:
(a) a first mechanical output from a usage detector,
(b) a first optical output from the usage detector,
(c) a first electrical output from the usage detector, or
(d) any combination of (a), (b), or (c).

16. The method of claim 15, the method further comprising disabling an LED (light emitting diode) within the endoscope.

17. The method of claim 15, wherein the usage detector device is configured to detect an initiation of use of the endoscope.

18. The method of claim 15, wherein the usage detector device is configured to detect a termination of use of the endoscope.

19. The method of claim 15, wherein the usage detector device is configured to detect a cable insertion and/or removal.

20. The method of claim 15, wherein the usage detector device detects a switch activation.

21. The method of claim 15, the method further comprising disabling the endoscope by at least partly obscuring an optical element.

22. The method of claim 15, the method further comprising disabling the endoscope by inhibiting illumination of a light source.

23. The method of claim 15, the method further comprising disabling the endoscope via an electrically controllable optical light transmission element.

24. The method of claim 15, the method further comprising disabling the endoscope via an electrically controllable optical light transmission element including an electro-chromatic layer and/or a liquid crystal.

25. The method of claim 15, the method further comprising disabling the endoscope via a fuse.

26. The method of claim 15, the method further comprising disabling the endoscope after a single use.

27. The method of claim 15, the method further comprising disabling the endoscope by disabling a light emission device, an image sensor, and/or a power source.

28. The method of claim 15, the method further comprising disabling the endoscope without using a sterilization process.

29. A method for manufacturing an endoscope for viewing portions of a body, the method comprising:
disposing on at least one portion of an endoscope body a usage detector device configured to detect a usage event related to the endoscope, the usage event including one or more of:
(i) an initiation of use of the endoscope,
(ii) a termination of use of the endoscope,
(iii) a cable insertion to the endoscope,
(iv) a cable removal from the endoscope,
(v) a switch activation, or
any combination of (i), (ii), (iii), (iv), or (v); and
disposing on at least one portion of an endoscope body a disabling device configured to permanently inhibit the use of the endoscope at least partly in response to:
(a) a first mechanical output from a usage detector,
(b) a first optical output from the usage detector,
(c) a first electrical output from the usage detector, or
(d) any combination of (a), (b), or (c).

30. The method of claim 29, wherein the disabling device is configured to disable a solid state light emission device.

31. The method of claim 29, wherein the usage detector device is configured to detect an initiation of use of the endoscope.

32. The method of claim 29, wherein the usage detector device is configured to detect a termination of use of the endoscope.

33. The method of claim 29, wherein the usage detector device is configured to detect a cable insertion and/or removal.

34. The method of claim 29, wherein the usage detector device is configured to detect a switch activation.

35. The method of claim 29, wherein the disabling device is configured to inhibit use of the endoscope by at least partly obscuring an optical element.

36. The method of claim 29, wherein the disabling device is configured to inhibit use of the endoscope by inhibiting illumination of a light source.

37. The method of claim 29, wherein the disabling device is configured to inhibit use of the endoscope via an electrically controllable optical light transmission element.
